(19) United States
(12) Patent Application Publication
(10) Pub. No.: US 2006/0188875 A1
(43) Pub. Date: Aug. 24, 2006

(54) Human genomic polymorphisms

(75) Inventors: David R. Cox, Belmont, CA (US); Nila Patil, Woodside, CA (US); Anthony J. Berno, San Jose, CA (US); David A. Hinds, Mountain View, CA (US)

Correspondence Address: PERLEGEN SCIENCES, INC.;LEGAL DEPARTMENT, 2021 STIERLIN COURT, MOUNTAIN VIEW, CA, 94043, US

(73) Assignee: Perlegen Sciences, Inc., Santa Clara, CA (US)

(21) Appl. No.: 10/284,444

(22) Filed: Oct. 31, 2002

Related U.S. Application Data
(63) Continuation of application No. 10/166,341, filed on Sep. 18, 2001, now abandoned.

(51) Int. Cl. C12Q 1/68; G06F 19/00; C07H 21/04

(52) U.S. Cl. 435/6; 536/24.3; 702/20

(57) ABSTRACT

The invention provides nucleic acid segments of the human genome including polymorphic sites, SNP haplotype blocks, SNP haplotype patterns for each block and informative SNPs for each pattern. Allele-specific primers and probes hybridizing to regions flanking these sites are also provided. The nucleic acids, primers and probes are used in applications such as association studies and other genetic analyses.

This application incorporates by reference herein in their entirety files submitted on duplicate compact discs, created Sep. 18, 2001 and containing the following files:

| File Name | Date Created | Size | |
|---|---|---|---|
| DbSNP.txt | Aug. 30, 2001 | 12.1 | MB |
| Group.txt | Aug. 30, 2001 | 398 | KB |
| Polymorphism_group_map.txt | Aug. 30, 2001 | 782 | KB |
| Pattern.txt | Aug. 30, 2001 | 636 | KB |

The file contents of these files are as follows:
dbSNP.txt:

Single Nucleotide Polymorphism data in dbSNP submission format: this specifies Perlegen's polymorphism ID code, a Genbank ID for the sequence, the position for the SNP in that sequence, 100 base pairs of sequence flanking the SNP, and the observed alleles at the SNP site.

group.txt:

For each haplotype group, this gives some general parameters that describe the group: a sequence ID code, the number of SNP's in the group and the minimum number of SNP's required to distinguish between the common patterns, starting and ending positions, and a range of polymorphism indexes included in the group.

polymorphism_group_map.txt:

This maps the polymorphism index values used in group.txt to the polymorphism ID codes used in dbSNP.txt.

pattern.txt:

This lists the observed haplotype patterns for all of the groups, and the number of samples that matched each pattern. Except for dbSNP.txt, all the files are tab-separated, with field names specified on the first line of the file.

Pertinent to the files disclosed on the accompanying CD that are incorporated by reference herein in their entirety, the following table maps sequence ID codes to sequence names composed of the chromosome number and Genbank identifier for the sequence.

| sequence_id | sequence_name |
|---|---|
| 2 | C21_NT_002836 |
| 4 | C21_NT_001035 |
| 8 | C21_NT_002835 |
| 12 | C21_NT_003545 |

### BACKGROUND OF THE INVENTION:

The genomes of all organisms undergo spontaneous mutation in the course of their continuing evolution generating variant forms of progenitor sequences (Gusella, Ann. Rev. Biochem. 55, 831-854 (1986)). The variant form may confer an evolutionary advantage or disadvantage relative to a progenitor form or may be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. In many instances, both progenitor and variant form(s) survive and co-exist in a species population. The coexistence of multiple forms of a sequence gives rise to polymorphisms.

Several different types of polymorphisms have been reported. A restriction fragment length polymorphism (RFLP) means a variation in DNA sequence that alters the length of a restriction fragment as described in Botstein et al., Am. J. Hum. Genet. 32, 314-331 (1980). The restriction fragment length polymorphism may create or delete a restriction site, thus changing the length of the restriction fragment. RFLPs have been widely used in human and animal genetic analyses (see WO 90/13668; WO90/11369; Donis-Keller, Cell 51, 319-337 (1987); Lander et al., Genetics 121, 85-99 (1989)). When a heritable trait can be linked to a particular RFLP, the presence of the RFLP in an individual can be used to predict the likelihood that the individual will also exhibit the trait.

Other polymorphisms take the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307, 113-115 (1992); Hom et al., WO 91/14003; Jeffreys, EP 370,719), and in a large number of genetic mapping studies.

Other polymorphisms take the form of single nucleotide variations between individuals of the same species. Such polymorphisms are far more frequent than RFLPs, STRs and VNTRs. Some single nucleotide polymorphisms (SNPs) occur in protein-coding sequences, in which case one of the polymorphic forms may give rise to the expression of a defective or other variant protein and, potentially, a genetic disease. Examples of genes in which polymorphisms within coding sequences give rise to genetic disease include β-globin (sickle cell anemia) and CFTR (cystic fibrosis). Other SNPs occur in noncoding regions. Some of these polymorphisms may also result in defective protein expression (e.g., as a result of defective splicing or faulty regulation of expression). Other SNPs have no phenotypic effects.

SNPs can be used in the same manner as RFLPs and VNTRs but offer several advantages. SNPs occur with greater frequency and are spaced more uniformly throughout the genome than other forms of polymorphism. The greater frequency and uniformity of SNPs means that there is a greater probability that such a polymorphism will be found in close proximity to a genetic locus of interest than would be the case for other polymorphisms. Also, the different forms of characterized SNPs are often easier to distinguish than other types of polymorphism (e.g., by use of assays employing allele-specific hybridization probes or primers).

Despite the increased amount of nucleotide sequence data being generated in recent years, only a fraction of the total repository of polymorphisms in humans and other organisms has been identified. The paucity of polymorphisms identified is due to the large amount of work required for detection by conventional methods. For example, a conventional approach to identifying polymorphisms might be to sequence the same stretch of oligonucleotides in a population of individuals by dideoxy sequencing. In this type of approach, the amount of work increases in proportion to both the length of sequence and the number of individuals in a population and becomes impractical for large stretches of DNA or large numbers of persons.

### SUMMARY OF THE CLAIMED INVENTION

The present invention provides nucleic acid segments of human chromosome 21 that include single nucleotide polymorphic sites. Allele-specific primers and probes hybridizing to regions flanking these sites are also provided, as are SNP haplotype blocks, SNP haplotype patterns for each block and informative SNPs for each pattern. The nucleic acids, primers and probes may be used in applications such as association studies and other genetic analyses such as disease diagnosis and expression analysis. Specifically, the present invention provides a nucleic acid segment of between 10 and 201 contiguous bases selected from the sequences in dbSNP.txt, where the nucleic acid segment may include either base at the SNP locus indicated. In addition, complements of this nucleic acid segment are provided, as are allele-specific oligonucleotides that hybridize to these nucleic acid segments and complements. Further, the present invention provides at least one SNP haplotype block from human chromosome 21 as described in group.txt, and at least one SNP haplotype pattern from a SNP haplotype block as described in pattern.txt.

### BRIEF DESCRIPTION OF THE FIGURES

The following figures and drawings form part of the present specification and are included to demonstrate certain aspects of the patent invention. The invention may be better understood by reference to one or more of these drawings in combination with the detailed description of the specific embodiments presented herein.

FIG. 1 includes a table with results obtained from screening hamster-human cell hybrids with the HuSNP GENECHIP° from Affymetrix, Inc. In addition, FIG. 1 shows the pattern of hybridization obtained when DNA samples from two hemizygous hybrid cell lines (A and B) and a diploid, heterozygous cell line (CDP17) are interrogated with a HuSNP chip.

FIG. 2 discloses various logistics of analyzing human chromosome 21 SNPs.

FIG. 3 shows the distribution of lengths of the SNP haplotype blocks found for human chromosome 21.

FIG. 4 shows the haplotype patterns for twenty independent globally diverse chromosomes defined by 147 common human chromosome 21 SNPs. The 147 SNPs span 106 kb of genomic DNA sequence.

FIG. 5 depicts the number of SNPs required to capture the common haplotype information for chromosome 21.

### DEFINITIONS:

An oligonucleotide can be DNA or RNA, and single- or double-stranded. Oligonucleotides can be naturally occurring or synthetic, but are typically prepared by synthetic means. Preferred oligonucleotides of the invention include segments of DNA, or their complements including any one of the polymorphic sites shown in the Listings on the attached disk. The segments are usually between 5 and 100 contiguous bases, and often between 5-10, 5-20, 10-20, 10-50, 15-50, 15-100, 20-50 or 20-100 contiguous bases. The polymorphic site can occur within any position of the segment. The segments can be from any of the allelic forms of DNA shown in file dbSNP.txt on the attached disk.

Hybridization probes are oligonucleotides capable of binding in a base-specific manner to a complementary strand of nucleic acid. Such probes include peptide nucleic acids, as described in Nielsen et al., Science 254, 1497-1500 (1991).

The term primer refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′ downstream primer that hybridizes with the complement of the 3′ end of the sequence to be amplified.

Linkage describes the tendency of genes, alleles, loci or genetic markers to be inherited together as a result of their location on the same chromosome.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

A single nucleotide polymorphism or SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). SNPs are most frequently diallelic. A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Hybridizations are usually performed under stringent conditions, for example, at a salt concentration of no more than 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations.

An isolated nucleic acid means an object species invention that is the predominant species present (i.e., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

Linkage disequilibrium or allelic association means the preferential association of a particular allele or genetic marker with a specific allele or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be used detected to indicate susceptibility to the disease in circumstances in which the gene Y may not have been identified or may not be readily detectable.

The present invention includes the use of any of the polymorphic forms, SNP haplotype blocks or SNP haplotype patterns shown in files dbSNP.txt, group.txt, pattern.txt of the CD ROM attached hereto as a means to determine susceptibility to a phenotype resulting from an allele or marker in linkage disequilibrium with such polymorphic forms.

Description

Polymorphisms of the Invention

The polymorphisms in files dbSNP.txt are SNPs from human chromosome 21. The files group.txt and pattern.txt are the SNP blocks and the SNP patterns within each SNP block that were determined by analyzing the common SNPs in dbSNP.txt. Common SNPs were defined as those SNPs that are present in at least 10% of the human population and that result from mutations that occurred early in the evolution of humans.

Systematic allele differences between control and experimental populations may appear as disease or drug-response associated, yet may result only from evolutionary or migratory history or mating practices. Focusing on common SNPs decreases the false positives that result from recent population anomalies and insufficient divergence time. Further, common SNPs are relevant to a larger proportion of the human population, making the SNPs used in the haplotype blocks and patterns of the present invention more broadly applicable to disease and drug response studies. However, the present invention also includes the rare SNPs in file dbSNP.txt. Rare SNPs are useful for studying individuals in a population, recent evolutionary or migratory history of populations, environmental effect on genetics of a population and the like.

The SNP haplotype blocks represent human chromosome 21 sequences containing groups of one to over one hundred SNPs that do not recombine independently but are passed from generation to generation in variable-length blocks. The set of genotypes for all the SNPs in a SNP haplotype block in a particular individual is a SNP haplotype pattern. If SNP haplotype patterns were random, it would be expected that the number of possible SNP haplotype patterns observed for a SNP haplotype block of N SNPs would be 2^{N}, but it was observed that the number of SNP haplotype patterns in each SNP haplotype block is smaller. In fact, it was found that generally 3 or 4 SNP haplotype patterns account for over 80% of the genotypes in each SNP haplotype block.

Informative SNPs are single SNPs or a subset (more than one) of SNPs which have been selected from the set of all SNPs in a SNP haplotype pattern to distinguish that SNP haplotype pattern from other SNP haplotype patterns in a specific SNP haplotype block. Thus, once SNP haplotype patterns for a particular SNP haplotype block are known, one can select an informative SNP or couple of SNPs from each SNP haplotype pattern to 1) identify the genotype of all other SNPs in that SNP haplotype pattern, and 2) distinguish the SNP haplotype pattern from other SNP haplotype patterns that belong to a particular SNP haplotype block.

The novel polymorphisms, SNP blocks and SNP patterns for human chromosome 21 of the invention are in files dbSNP.txt, group.txt, and pattern.txt of the CD ROM accompanying this patent application. Files sequence.txt and polymorphism_group_map.txt are files that link the information in dbSNP.txt, group.txt, and pattern.txt to each other or to sequences in GenBank. Thus, the attached CD ROM contains five files.

File dbSNP.txt contains human chromosome 21 SNP data, specifying a Perlegen Sciences ID code, a GenBank Accession nucleotide number, and the number of samples tested. In addition, for each sequence, the position of the SNP in that sequence (indicated at COMMENT), 100 base pairs of sequence 5′ to the SNP, 100 base pairs of sequence 3′ of the SNP, and the observed two alleles at the site of the SNP are shown.

File group.txt contains information for each haplotype group on human chromosome 21, including two columns of Perlegen Sciences group ID codes, a Perlegen group analysis ID reflecting the parameters used to determine SNP haplotype blocks, a Perlegen sequence ID number (see the description of sequence.txt, below), the number of SNPs required to distinguish between the common SNP haplotype patterns in each SNP haplotype block (that is, the number of informative SNPs required for that block), the informativeness of each block (the total number of SNPs in each block divided by the number of informative SNPs required to distinguish the SNP haplotype patterns in each SNP haplotype block), a first and last polymorphism index number (see the description of polymorphism_group_map.txt, below), and reference positions within the GenBank sequence (again, please see the description of sequence.txt, below).

File pattern.txt lists the observed common SNP haplotype patterns for all SNP haplotype blocks and the number of samples that matched each pattern. The file lists a Perlegen group ID number (see group.txt and polymorphism_group_map.txt), a Perlegen assigned pattern ID number, the sequence of the pattern and the number of samples tested that had this pattern.

File polymorphism_group_map.txt lists the Perlegen group ID number, a Perlegen polymorphism ID number assigned to a SNP when it is first identified, and a Perlegen polymorphism index number assigned to a SNP for haplotype analysis. Every SNP identified was assigned a polymorphism ID number, including rare SNPs. However, only common SNPs used in haplotype analysis were assigned a polymorphism index number. Note that the polymorphism index number is one of the columns in group.txt.

Finally, file sequence.txt is a table with columns of “sequence_ID” and “sequence_name”. The sequence_ID column is a Perlegen assigned sequence ID (which is also the fourth column in group.txt). The sequence name column gives the GenBank sequence identifier.

Analysis of Polymorphisms

Polymorphisms are detected in a target nucleic acid from an individual being analyzed. For an assay of genomic DNA, virtually any biological sample (other than pure red blood cells) is suitable. For example, convenient tissue samples include whole blood, semen, saliva, tears, urine, fecal material, sweat, buccal, skin and hair. For assays of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes). Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Detecting polymorphisms focuses on comparing sequences in different individuals to identify points of variation, i.e., polymorphic sites. By analyzing groups of individuals, frequencies of variation at each SNP locus (allelic frequency) and in haplotype patterns in a population can be determined. Once a baseline of allelic/haplotype pattern frequencies is determined for a population, allelic/haplotype pattern frequencies can be determined for subpopulations characterized by criteria such as geography, race, gender, disease susceptibility or response to therapeutics.

Polymorphisms can be detected using allele-specific probes. The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

Polymorphisms can also be identified by hybridization to nucleic acid arrays, some examples of which are described by WO 95/11995 (incorporated by reference in its entirety for all purposes). One form of such arrays is described in the Example 4 in connection with identification of polymorphisms. WO 95/11995 also describes subarrays that are optimized for detection of variant forms of precharacterized polymorphisms. Such a subarray contains probes designed to be complementary to a second reference sequence, which is an allelic variant of the first reference sequence. The second group of probes is designed by the same principles except that the probes exhibit complementarity to the second reference sequence. The inclusion of a second group (or further groups) of probes can be particularly useful for analyzing short subsequences of the primary reference sequence in which multiple mutations are expected to occur within a short distance commensurate with the length of the probes (i.e., two or more mutations within 9 to 21 bases).

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers leading to a detectable product indicating whether the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′-most position of the oligonucleotide aligned with the polymorphism because this position is most destabilizing to elongation from the primer. See, e.g., WO 93/22456.

The direct analysis of the sequence of polymorphisms of the present invention can be accomplished using either the dideoxy-chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

Methods of Use

The polymorphisms of the present invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. By analogy, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation, i.e. regulatory effects. A single polymorphism may affect more than one phenotypic trait. Likewise, a single phenotypic trait may be affected by polymorphisms in different genes.

Phenotypic traits include symptoms of, or susceptibility to, diseases of which one or more components is or may be genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, color obesity), strength, speed, endurance, fertility, and susceptibility or receptivity to particular drugs or therapeutic treatments. Many human disease phenotypes can be simulated in animal models. Examples of such models include inflammation (see e.g., Ma, Circulation 88, 649-658 (1993); multiple sclerosis (Yednock et al., Nature 356, 63-66 (1992); Alzheimer's disease (Games, Nature 373, 523 (1995); Hsiao et al., Science 250, 1587-1590 (1990)); cancer (see Donehower (1992) Nature 356, 215 Clark (1992) Nature 359, 328; Jacks (1992) Nature 359, 295; and Lee (1992) Nature 359, 288; cystic fibrosis (Snouwaert (1992) Science 257, 1083); Gaucher's Disease (Tybulewicz (1992) Nature 357, 407); hypercholesterolemia (Piedrahita (1992) PNAS 89, 4471); neurofibromatosis (Brannan (1994) Genes & Dev. 7, 1019); Thalaemia (Shehee (1993) PNAS 90, 3177); Wilm's Tumor (Kreidberg (1993) Cell 74, 679); DiGeorge's Syndrome (Chisaka (1991) Nature 350, 473); infantile pyloric stenosis (Huang (1993) Cell 75, 1273); inflammatory bowel disease (Mombaerts (1993) Cell 75, 275).

Correlation is performed for a population of individuals who have been tested for the presence or absence of a phenotypic trait of interest and for polymorphic markers sets. To perform such analysis, the presence or absence of a set of polymorphisms (i.e. a polymorphic set) is determined for a set of the individuals, some of whom exhibit a particular trait, and some of whom exhibit lack of the trait. The alleles of each polymorphism of the set are then reviewed to determine whether the presence or absence of a particular allele is associated with the trait of interest. Correlation can be performed by standard statistical methods and statistically significant correlations between polymorphic form(s) and phenotypic characteristics are then noted. For example, it might be found that the presence of allele A1 at polymorphism A correlates with heart disease. As a further example, it might be found that the combined presence of allele A1 at polymorphism A and allele B1 at polymorphism B correlates with increased milk production of a farm animal.

Such correlations can be exploited in several ways. Correlations established between animal phenotypes and polymorphic form(s) can be retested for cognate human phenotypes and polymorphic forms. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic set in a patient correlated with enhanced receptiveness to one of several treatment regimes for a disease indicates that this treatment regime should be followed.

In addition to identifying correlations between phenotypic traits and polymorphisms that directly or indirectly contribute to those traits, one also may identify physical linkage between a genetic locus associated with a trait of interest and polymorphic markers that are not associated with the trait, but that are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992) (each of which is incorporated by reference in its entirety for all purposes).

The SNP blocks and patterns of the present invention are useful for predicting genomic locations of disease-related or drug response-related genes. Such predictions involve determining SNP haplotype patterns from individuals in a control population where the haplotype patterns occur with a certain frequency in the control population. Next, the frequency of SNP haplotype patterns is determined from a clinical population who are diseased or to whom a drug has been administered. A comparison is then made between the frequencies of SNP haplotype patterns that occur in these two populations. SNP haplotype patterns that occur in a higher or lower than average frequency in the clinical population indicate areas in the genome where disease-related or drug-response related genes or genetic loci are located. Once informative SNPs are determined, the frequency of occurrence of informative SNPs for each SNP haplotype pattern in the control and clinical populations may be used to determine the location of disease-related or drug-related genetic loci, thereby decreasing the number of genetic loci that need to be examined.

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage may be analyzed by calculation of LOD (log of the odds) values. A lod value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W.B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log10 of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of 0 (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

Associations of variations with other variations, e.g., SNPs with each other in a SNP haplotype block, also may be determined by computational methods based on observed, empiric sequence variations in several individuals using genome scanning of a genetic locus. Sequences from different origins may be compared, SNPs scored, and a SNP map constructed. Once the individual SNPs are determined, the SNP haplotype blocks and SNP haplotype patterns within the SNP haplotype blocks can be defined. In one example, an algorithm then was used to identify an informative SNP for each SNP haplotype pattern. The informative SNP was selected so that the genotype of the informative SNP predicts the genotype of the remaining SNPs in that SNP haplotype pattern. Knowing the informative SNPs for all patterns in all SNP haplotype blocks allows for the design of less expensive genotyping assays that retain most of the power of an assay constructed using all SNPs.

Modified Polypeptides and Gene Sequences

The invention further provides forms of nucleic acids and corresponding proteins. The nucleic acids comprise one or more of the sequences described in file dbSNP.txt, in which the polymorphic position is occupied by one of the alternative bases noted for that position. Some of the alternative SNPs or variations occur in coding regions and account for variant forms of proteins. Proteins may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

The invention further provides transgenic nonhuman animals capable of expressing an exogenous gene and/or having one or both alleles of an endogenous gene inactivated. Expression of an exogenous gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory. Inactivation of endogenous genes can be achieved by forming a transgene in which a cloned variant gene is inactivated by insertion of a positive selection marker. See Capecchi, Science 244, 1288-1292 (1989). The transgene is then introduced into an embryonic stem cell, where it undergoes homologous recombination with an endogenous gene. Mice and other rodents are preferred animals. Such animals provide useful drug screening systems.

In addition to substantially full-length polypeptides expressed by genes, the present invention includes biologically active fragments of the polypeptides, or analogs thereof, including organic molecules that simulate the interactions of the peptides. Biologically active fragments include any portion of the full-length polypeptide which confers a biological function on the variant gene product, including ligand binding, and antibody binding. Ligand binding includes binding by nucleic acids, proteins or polypeptides, small biologically active molecules, or large cellular structures.

Polyclonal and/or monoclonal antibodies that specifically bind to variant gene products but not to corresponding prototypical gene products are also provided. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

Antibodies which bind specifically to the gene products of the present invention may be used for the diagnosis of disorders characterized by their expression or in assays to monitor patients being treated with the gene products or with agonists, antagonists or inhibitors of the gene products. Diagnostic assays for the gene products of the present invention include methods which utilize the antibody and a label to detect the gene product in human body fluids or in extract of cells or tissues. The antibodies may be used with or without modification, and may be labeled by covalent or non-covalent attachment of a reporter molecule.

Additionally, polynucleotides encoding the gene products of the present invention may be used for diagnostic purposes. The polynucleotides which may be used include oligonucleotide sequences, complementary RNA and DNA molecules and PNAs. The polynucleotides may be used to detect and quantitate gene expression in tissues in which expression of the gene product may be correlated with disease. The diagnostic assay may be used to determine absence, presence, and excess expression of the gene product, or to monitor regulation of gene product levels during therapeutic intervention. Also, the polynucleotide sequences of the present invention may be used for the diagnosis of a disorder associated with a genetic locus proximal to the polynucleotide sequence. The polynucleotide sequences may be used in Southern or Northern analysis, dot blot, or other membrane based technologies, in PCR technologies, in dipstick assays, and in microarrays utilizing fluids or tissue extracts from patients. The polynucleotide sequences of the present invention, and longer or shorter sequences derived therefrom, also may be used as targets in a microarray or other genotyping system. These systems can be used to detect the presence or absence of a large number of particular allelic SNP forms or to monitor the expression of a large number of gene products simultaneously.

In another embodiment of the present invention, the polynucleotides, or any fragment or complement thereof, may be used for therapeutic purposes. In one aspect, the complement of the polynucleotide may be used in situations in which it would be desirable to block the transcription of mRNA. In particular, cells may be transformed with sequences complementary to polynucleotides encoding the gene products of the present invention. Thus, the complementary molecules may be used to modulate gene product activity or to achieve regulation of gene function. Sense and antisense oligonucleotides may be designed from the polynucleotide sequences of the present invention. Polynucleotides may be introduced into an organism by methods known in the art, and genes can be expressed in an expression vector in which the gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing an expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like. An additional embodiment of the invention relates to the administration of a pharmaceutical or sterile composition, in conjunction with a pharmaceutically acceptable carrier, for any of the therapeutic effects discussed herein. Such pharmaceutical compositions may consist of the gene products of the present invention, antibodies to the gene products, mimetics, agonists, antagonists or inhibitors to the gene products.

Kits

The invention further provides kits comprising at least one allele-specific oligonucleotide as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 10, 100 or all of the polymorphisms listed in dbSNP.txt. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

Computer Systems for Storing Polymorphism Data

A computer system suitable for storing the files of the present invention may include a bus which interconnects major subsystems such as a central processor, a system memory (typically RAM), an input/output (I/O) controller, an external device such as a display screen via a display adapter, serial ports, a keyboard, a fixed disk drive via a storage interface and a floppy disk drive operative to receive a floppy disk, and a CD-ROM (or DVD-ROM) device operative to receive a CD-ROM. Many other devices may be connected such as a user pointing device, e.g., a mouse connected via a serial port, and a network interface connected via another serial port. Many other devices or subsystems may be connected in a similar manner. Also, it is not necessary for all of the components described to be present to practice the present invention. Further, the devices and subsystems may be interconnected in different ways. The operation of a computer system such as that described is well known. Databases storing polymorphism information according to the present invention can be stored, e.g., in a system memory or on storage media such as a fixed disk, floppy disk, or CD-ROM. An application program to access such databases can be operably disposed in a system memory or sorted on storage media such as a fixed disk, floppy disk, or CD-ROM.

In addition, the computer system may be interconnected with other remote computers in a network with interconnecting remote servers. The network interface can provide connection from the client computer system to the network. The network can be e.g., the Internet. Protocols for exchanging data via the Internet and other networks are well known. Information identifying the polymorphisms described herein can be transmitted across the network and embedded in signals capable of traversing the physical media employed by the network.

Information identifying polymorphisms in each file is represented in records, which optionally, are subdivided into fields. Each record stores information relating to a different polymorphisms. Collectively, the records can store information relating to all of the polymorphisms, or any subset thereof, such as 5, 10, 50, or 100 polymorphisms from dbSNP.txt. In some databases, the information identifies a base occupying a polymorphic position and the location of the polymorphic position. The base can be represented as a single letter code (i.e., A, C, G or T/U) present in a polymorphic form other than that in the reference allele. Alternatively, the base occupying a polymorphic site can be represented in IUPAC ambiguity code. The location of a polymorphic site can be identified as its position within one of the sequences shown in file dbSNP.txt and reference sequences in the public database GenBank. For example, in the sequences in dbSNP.txt, the polymorphic site occupies the 101st base and the 100 base pairs 5′ and 100 base pairs 3′ to each polymorphic site is given. The position can also be identified by reference to, for example, a chromosome, and distance from known markers within the chromosome. In other databases, information identifying a polymorphism contains sequences of 10-100 bases from dbSNP.txt or the complements thereof.

### EXAMPLES

### Example 1

### Preparations of Somatic Cell Hybrids

Standard procedures in somatic cell genetics were used to separate human DNA strands (chromosomes) from a diploid state to a haploid state. For example, diploid human lymphoblast cell lines were fused to a diploid hamster fibroblast cell line containing a mutation in the thymidine kinase gene. In a sub-population of the resulting fused cells, human chromosomes were introduced into the hamster calls. Selection for the human DNA-containing hamster cells (fusion cells) was achieved by utilizing HAT medium. Only hamster cells that had a stably incorporated human DNA strand grow in cell culture medium containing HAT.

Hamster cell line A23 cells were pipetted into a centrifuge tube containing 10 ml DMEM in which 10% FBCS+1× Pen/Strep+10% glutamine were added, centrifuged at 1500 rpm for 5 minutes, resuspended in 5 ml of RPMI and pipetted into a tissue culture flask containing 15 ml RPMI medium. The lymphoblast cells were grown at 37° C. to confluence. At the same time, human lymphoblast cells were pipetted into a centrifuge tube containing 10 ml RPMI in which 15% FBCS+1× Pen/Strep+10% glutamine were added, centrifuged at 1500 rpm for 5 minutes, resuspended in 5 ml of RPMI and pipetted into a tissue culture flask containing 15 ml RPMI. The lymphoblast cells were grown at 37° C. to confluence.

To prepare the A23 hamster cells, the media was aspirated and the cells were rinsed with 10 ml PBS. The cells were then trypsinized with 2 ml of trypsin and divided into 3-5 plates of fresh media (DMEM without HAT) and incubated at 37° C. The lymphoblast cells were prepared by transferring the culture into a centrifuge tube and centrifuging at 1500 rpm for 5 minutes, resuspending the cells in 5 ml RPMI and pipetting 1 to 3 ml of cells into 2 flasks containing 20 ml RPMI.

To achieve cell fusion, approximately 8-10×10⁶ lymphoblast cells were centrifuged at 1500 rpm for 5 min. The cell pellet was then rinsed with DMEM by resuspending the cells and centrifuging them again. The lymphoblast cells were then resuspended in 5 ml DMEM. The recipient A23 hamster cells had been grown to confluence and split 3-4 days before the fusion and were, at this point, 50-80% confluent. The old media was removed and the cells were rinsed 3 times with DMEM and finally suspended in 5 ml DMEM. The lymphoblast cells were slowly pipetted over the recipient A23 cells and the combined culture was swirled slowly before incubating at 37° C. for 1 hour. After incubation, the media was gently aspirated from the A23 cells, and 2 ml room temperature PEG 1500 was added by touching the edge of the plate with a pipette and slowly adding PEG to the plate while rotating the plate with the other hand. It took approximately one minute to add all the PEG in one full rotation of the plate. Next, 8 ml DMEM was added down the edge of the plate while rotating the plate slowly. The PEB/DMEM mixture was aspirated gently from the cells and then 8 ml DMEM was used to rinse the cells. This DMEM was removed and 10 ml fresh DMEM was added and the cells were incubated for 30 min. at 37° C. Again the DMEM was aspirated from the cells and 10 ml DMEM in which 10% FBCS and 1× Pen/Strep were added, was added to the cells, which were then allowed to incubate overnight.

After incubation, the media was aspirated and the cells were rinsed with PBS. The cells were then trypsinized and divided among 20 plates containing selection media (DMEM in which 10% FBCS+1× Pen/Strep+1×HAT were added) so that each plate received approximately 100,000 cells. The media was changed on the third day following plating. Colonies were picked and placed into 24-well plates upon becoming visible to the naked eye (day 9-14). If a picked colony was confluent within 5 days, it was deemed healthy and the cells were trypsinized and moved to a 6-well plate.

DNA and stock hybrid cell cultures were prepared from the cells from the 6-well plate cultures. The cells were trypsinized and divided between a 100 mm plate containing 10 ml selection media and an ependorf tube. The cells in the tube were pelleted, resuspended 200 μl PBX and DNA was isolated using a Qiagen DNA mini kit at a concentration of <5 million cells per spin column. The 100 mm plate was grown to confluence, and the cells were either continued in culture or frozen.

### Example 2

### Selecting Haploid Hybrids

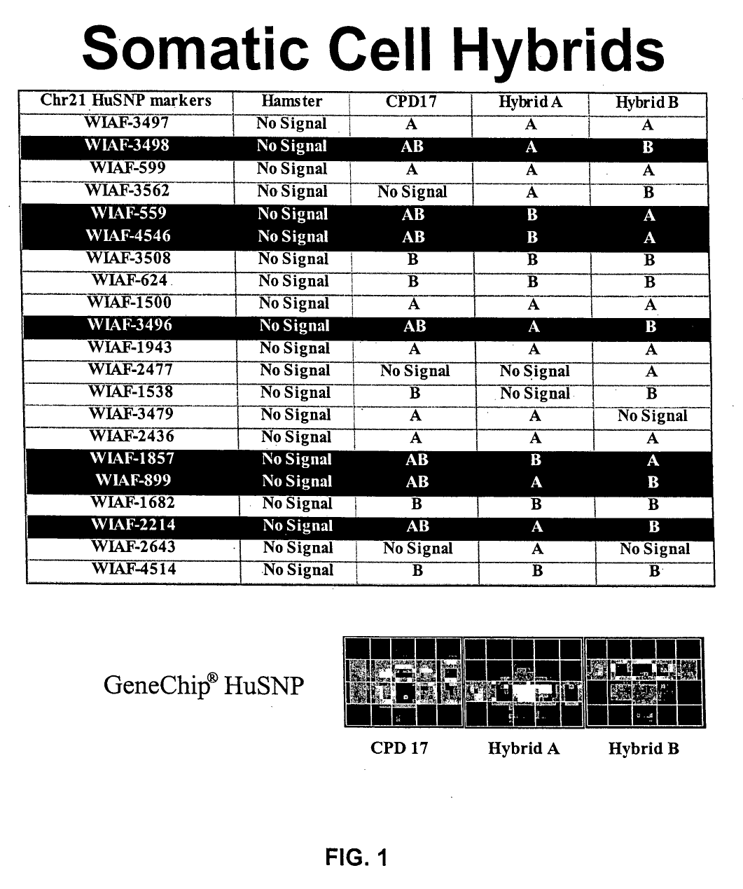

Scoring for the presence, absence and diploid/haploid state of each hybrid was performed using the Affymetrix, Inc. HuSNP GENECHIP® (Affymetrix, Inc. of Santa Clara, Calif., GENECHIP® HuSNP Mapping Assay, reagent kit and user manual, Affymetrix Part No. 900194), which can score 1494 markers in a single chip hybridization. As a control, the human diploid lymphoblast cell line was screened using the HuSNP chip hybridization assay, and any SNPs which were heterozygous in the parent lymphoblast diploid cell line were scored for haploidy in each fusion cell line. By comparing the markers that were present as “AB” heterozygous in the parent diploid cell line to the same markers present as “A” or “B” (hemizygous) in the hybrids, the human DNA strands which were in the haploid state in each hybrid line was determined. FIG. 1 has a table with a portion of results obtained by screening hamster-human cell hybrids with the HuSNP GENECHIP®. One column of the table lists SNP markers from human chromosome 21. The other columns list the results of the screen with hamster cell non-hybrids, a diploid non-hybrid human lymphoblast cell line, and two different human-hamster cell line hybrids prepared by the methods described in Example 1. In addition, FIG. 1 shows an image of the hybridization fluorescence for the diploid human cell line and the hybrids. Note that the pattern of hybridization for the diploid cell line indicates heterozygosity for this SNP position, and the patterns for Hybrids A and B indicate that the hybrid cell lines are haploid for the SNP shown and have different nucleotide basis at this particular SNP location.

### Example 3

### Long Range PCR

DNA from the hamster-human cell hybrids was used to perform long-range PCR assays. The products of this PCR were used to determine the sequence of specific DNA strand regions from chromosome 21 of the 50 haploid human genomes, and SNPs were discovered and scored by comparing the individual sequences. Long range PCR assays are known generally in the art and have been described, for example, in the standard long range PCR protocol from the Boehringer Mannheim Expand™ Long Range PCR Kit and in U.S. Pat. No. 5,512,462 to Cheng.

Primers used for the amplification reaction were designed in the following way: a given sequence, for example the 23 megabase contig on chromosome 21, was entered into a software program known in the art herein called “repeat masker” which recognizes sequences that are repeated in the genome (i.e., Alu and Line elements). The repeated sequences were “masked” by the program by substituting each specific nucleotide of the sequence (A, T, G or C) with “N”. The sequence output after this repeat mask substitution was then fed into a commercially available primer design program (Oligo 6.23) to select primers that were greater than 30 nucleotides in length and had melting temperatures of over 65° C. The designed primer output from Oligo 6.23 was then fed into a program which then “chose” primer pairs which would PCR amplify a given region of the genome but have minimal overlap. The success rate for long range PCR using commercially available protocols and this primer design is >80%, and >95% success can be achieved.

An illustrative protocol for long range PCR uses the Expand™ Long Template PCR System from Boehringer Mannheim Cat.# 1681 834, 1681 842, or 1759 060. In the procedure each 50 μL PCR reaction requires two master mixes. In a specific example, Master Mix 1 was prepared for each reaction in 1.5 ml microfuge tubes on ice and includes a final volume of 19 μL of Molecular Biology Grade Water (Bio Whittaker, Cat.# 16-001Y); 2.5 μL 10 mM dNTP set containing dATP, dCTP, dGTP, and dTTP at 10 mM each (Life Technologies Cat.# 10297-018) for a final concentration of 400 μM of each dNTP; and 50 ng DNA template.

Master Mix 2 for all reactions was prepared and kept on ice. For each PCR reaction Master Mix 2 includes a final volume of 25 μL of Molecular Biology Grade Water (Bio Whittaker); 5 μL 10×PCR buffer 3 containing 22.50 mM MgCl₂ (Sigma, Cat.# M 10289); 2.5 μL 10 mM MgCl₂ (for a final MgCl₂ concentration of 2.75 mM); and 0.75 μL enzyme mix (added last).

Six microliters of premixed primers (containing 2.5 μL of Master Mix 1) was added to appropriate tubes, then 25 μL of Master Mix 2 was added to each tube. The tubes were capped, mixed, centrifuged briefly and returned to ice. At this point, the PCR cycling was begun according to the following program: step 1: 94° C. for 3 min to denature template; step 2: 94° C. for 30 sec; step 3: annealing for 30 sec at a temperature appropriate for the primers used; step 4: elongation at 68° C. for 1 min/kb of product; step 5: repetition of steps 2-4 38 times for a total of 39 cycles; step 6: 94° C. for 30 sec; step 7: annealing for 30 sec; step 8: elongation at 68° C. for 1 min/kb of product plus 5 additional minutes; and step 9: hold at 4° C. Alternatively, a two-step PCR would be performed: step 1: 94° C. for 3 min to denature template; step 2: 94° C. for 30 sec; step 3: annealing and elongation at 68° C. for 1 min/kb of product; step 4: repetition of steps 2-3 38 times for a total of 39 cycles; step 5:94° C. for 30 sec; step 6: annealing and elongation at 68° C. for 1 min/kb of product plus 5 additional minutes; and step 7: hold at 4° C. Results of the long range PCR amplification reaction were visualized on ethidium bromide-stained agarose gels.

### Example 4

### Wafer Design, Manufacture, Hybridization and Scanning

The set of oligonucleotide probes to be contained on the wafer was defined based on the human DNA strand sequence to be queried. The oligonucleotide sequences were based on consensus sequences reported in publicly available databases. Once the probe sequences were defined, computer algorithms were used to design photolithographic masks for use in manufacturing the probe-containing wafers. Probe wafers were manufactured by a light-directed chemical synthesis processes which combines solid-phase chemical synthesis with photolithographic fabrication techniques. See, for example, WO 92/10092, or U.S. Pat. Nos. 5,143,854; 5,384,261; 5,405,783; 5,412,087; 5,424,186; 5,445, 934; 5,744,305; 5,800,992; 6,040,138; 6,040,193, all of which are incorporated herein by reference in their entireties for all purposes. Using a series of photolithographic masks to define exposure sites on the wafer followed by specific chemical synthesis steps, the process constructed high-density arrays of oligonucleotide probes on the wafer, with each probe in a predefined position. Multiple probe regions were synthesized simultaneously and in parallel on the glass wafer.

The synthesis process involved selectively illuminating a photo-protected glass substrate (wafer) by passing light through a photolithographic mask wherein chemical groups in unprotected areas were activated by the light. The selectively-activated substrate wafers were then incubated with a chosen nucleoside, and chemical coupling occurred at the activated positions on the wafer. Once coupling took place, a new mask pattern was applied and the coupling step was repeated with another chosen nucleoside. This process was repeated until the desired set of probes was obtained. In one specific example, 25-mer oligonucleotide probes were used, where the thirteenth base was the base to be queried. Four probes were used to interrogate each nucleotide present in each sequence—one probe complementary to the sequence and three mismatch probes identical to the complementary probe except for the thirteenth base.

Once fabricated, the arrays were hybridized to the sample products from the long range PCR reactions performed on the hamster-human cell hybrids. The samples to be analyzed were labeled and incubated with the wafer to allow hybridization of the sample to the probes on the wafer.

After hybridization, the wafer was inserted into a confocal, high performance scanner, where patterns of hybridization were detected. The hybridization data were collected as light emitted from fluorescent reporter groups already incorporated into the PCR products of the sample, which was bound to the wafer probes. Probes that are complimentary to sequences present in the sample hybridized to the wafer more strongly and produced stronger signals than those sequences that had mismatches. Since the sequence and position of each probe on the array was known, by complementarity, the identity of the variation in the sample nucleic acid applied to the probe array was identified. Scanners and scanning techniques used in the present invention are known to those skilled in the art and are disclosed in, e.g., U.S. Pat. No. 5,981,956 drawn to microarray chips, U.S. Ser. No. 60/223,278 filed on Aug. 3, 2000, and the non-provisional application claiming priority to U.S. Ser. No. 60/223,278 which was filed on Aug. 3, 2001, drawn to scanners and techniques for whole wafer scanning, both references being incorporated herein by reference in their entireties for all purposes.

### Example 5

### Scoring SNPs, Determining SNP Haplotype Blocks and Patterns and Choosing Informative SNPs

Sequences from each DNA strand from different origins were compared, SNPs were scored, and a SNP map was constructed. Once the individual SNPs were determined, the SNP haplotype blocks and SNP haplotype patterns within the SNP haplotype blocks were defined. In one example, a greedy algorithm was used to identify an informative SNP for each SNP haplotype pattern. The informative SNP was selected so that the genotype of the informative SNP predicts the genotype of the remaining SNPs in that SNP haplotype pattern. Knowing the informative SNPs for all patterns in all SNP haplotype blocks allows for the design of less expensive genotyping assays that retain most of the power of an essay constructed using all SNPs.

The set of SNPs used to generate the haplotype blocks and haplotype patterns in files group.txt and pattern.txt in the present invention generally excluded singleton SNPs, or SNPs in which an allele was observed only once. In certain analyses, transition SNPs of the form Cg<-> Tg or cG <-> cA, were also excluded.

If SNP haplotype patterns were random, it would be expected that the number of possible SNP haplotype patterns observed for a SNP haplotype block of N SNPs would be 2^{N}, but it was observed that the number of SNP haplotype patterns in each SNP haplotype block is smaller. The degree of reduction in SNP haplotype pattern count was quantified by the number of non-singleton patterns that were observed in a particular block; that is, the number of patterns that contained more than one SNP, as well as the total number of samples that were associated with these patterns. The fraction of samples associated with non-singleton patterns as compared to the total number of samples is called the coverage of the block. The coverage indicates the expected number of patterns that, because of their relatively high frequency, could be predicted from the informative SNPs drawn from that block. Singleton patterns may be unique to the sample studied, and are therefore not considered when computing coverage.

Generally speaking, as many as 2^{N} distinct, non-singleton SNP haplotype patterns can be distinguished by using the genotypes of N suitably selected SNPs. Therefore, if there exist only two non-singleton SNP haplotype patterns in the SNP haplotype block, a single SNP could differentiate between the two. If there are three or four non-singleton patterns, at least two SNPs would be required, etc. The present invention utilized this standard to assign informative SNPs for each SNP haplotype pattern. Given a sufficient number of informative SNPs to distinguish between all non-singleton patterns, the existence of a particular pattern in an unknown sample was able to be inferred with an accuracy given by the coverage.

It was determined that informativeness is approximately inversely related to the information content, or entropy, of all genotypes in that block. In order to determine a useful informative subset for a large number of SNPs, a set of non-overlapping blocks that encompass all of the SNPs under consideration was determined. This set of non-overlapping blocks maximized the average informativeness of all the SNPs. Once this set of blocks was found, an informative SNP or subset of SNPs was drawn from each SNP haplotype pattern in each SNP haplotype block which was used to infer the genotypes of all the remaining SNPs with an accuracy determined by the block's coverage. By taking SNPs in order of decreasing informativeness, a trade-off can be established between the total number of SNPs used and the total number of SNPs predicted.

In order to find a set of non-overlapping blocks with optimal or near-optimal informativeness, those with skill in the art recognize a variety of algorithms can be used. A convenient and effective strategy is to use a greedy algorithm; however, one of skill in the art recognizes that other algorithms may be used, including a shortest path algorithm, and other algorithms known in the art, the goal being to attain the maximum amount of information from the least number of SNPs. A greedy algorithm begins by constructing a list of all possible SNP haplotype blocks subject to a minimal level of coverage. This list was sorted in descending order according to the informativeness of each block. Each candidate SNP haplotype block was evaluated in order, and if the block did not overlap with any previously selected block, it was selected as part of the data set.

Once all possible SNP haplotype blocks had been evaluated, the result was a set of non-overlapping SNP haplotype blocks that encompasses all the SNPs in the original set. Some blocks, called isolates, may consist of only a single SNP, and by definition have an informativeness of 1. Other blocks may consist of a hundred or more SNPs, and have an informativeness exceeding 30. Linkage disequilibrium mapping requires that a susceptibility allele be detectable with a marker that lies within the interval afforded by the SNP map density. One skilled in the art can construct such a map from the description provided herein.

The selection of at least 1 g(p) (log₂ p) SNPs from each block containing p non-singleton patterns (rounding up to the nearest integer) provides one set of SNPs which are unusually powerful for predicting genotype/phenotype associations. One skilled in the art recognizes that in other analyses it is not necessary to use spatially contiguous blocks to determine such a subset.

### Example 6

### Association of Disease-Related and Drug-Response Phenotypes with SNP Haplotype Blocks

The full set of DNA strands from 50 different origins (“control”) are separated, scored for SNPs, SNP haplotype blocks are assigned, and SNP haplotype patterns and the informative SNPs for each pattern are determined. Probes are then designed for microarrays that interrogate the 300,000-5000,000 informative SNPs. The approach of the present invention has tremendous advantage in conducting genetic association studies. Further, using the comprehensive SNP sequences of the present invention confers tremendous advantage over other whole genome scanning or genotyping methods known in the art; instead of reading all bases of each individual's chromosome 21—or even reading the common SNPs that may be found—only informative SNPs from the sample population need to be determined. The haplotype patterns seen by reading these particular, informative SNPs provide sufficient information to allow statistically accurate association data to be extracted from specific experimental populations. For example, collections of DNA samples from individuals from specific clinical populations are probed with informative SNP wafers to determine the genotype for each individual or for the block. The genotype for each individual of the block is determined by hybridization with informative SNPs; alternatively, DNA samples from the clinical population may be pooled and the pooled samples tested by hybridization. The informative SNPs determine the SNP haplotypes, which provide the location in the DNA strand of the relevant genetic targets. Thus, even multigenic associations are detected.

### Example 7

### Determination of SNP Haplotype Blocks and Patterns on Human Chromosome 21

Global patterns of human DNA sequence variation defined by common single nucleotide polymorphisms (SNPs) have important implications for all human traits. High-density oligonucleotide arrays were used in combination with somatic cell genetics to identify a large fraction of all common human chromosome 21 SNPs and to observe directly the haplotype structure defined by these SNPs. This structure reveals SNP haplotype blocks of limited SNP haplotype pattern diversity in which greater than 80% of a global human sample is characterized by only three common SNP haplotype patterns. Results indicate that 300,000 informative SNPs selected with prior knowledge of common SNP haplotype pattern structure across the genome will provide a powerful general reagent for whole-genome association analysis. Methods for identifying human haplotypes, haplotype blocks, haplotype patterns and informative variations may be found in U.S. Ser. No. 60/280,530 and in a provisional application filed Aug. 17, 2001 in the USPTO claiming priority to 60/280,530.

Human DNA sequence variation accounts for a large fraction of observed differences between individuals, including susceptibility to disease. The majority of human sequence variation is due to substitutions that occurred once in the history of mankind at individual base pairs. Although most SNPs are rare, it has been estimated that 5.3 million common SNPs, each with a frequency of 10-50%, account for the bulk of the DNA sequence difference between humans, and it is estimated that SNPs are present in the human genome once every 600 base pairs. Moreover, SNPs in close physical proximity are often linked, defining a “SNP haplotype”, and reflect descent from a single, ancient ancestral chromosome. While a SNP haplotype block of 10 independent biallelic SNPs could in theory generate 210 (approximately 1000) different SNP haplotype patterns, in the absence of recurrent mutation and/or recombination, the number of observed haplotypes should be less than 10 (i.e., the number of SNPs in the block).

The complexity of local haplotype structure in the human genome and the distance over which individual haplotypes extend is poorly defined. Empiric studies investigating different segments of the human genome in different populations have revealed tremendous variability in local haplotype structure. These studies indicate that the relative contributions of mutation, recombination, selection, population history and stochastic events to haplotype structure vary in an unpredictable manner, resulting in some haplotypes that extend for only a few kilobases (kb), and others that extend for greater than 100 kb. These findings suggest that any comprehensive description of the haplotype structure of the human genome, defined by common SNP haplotype patterns, requires empiric analysis of a dense set of SNPs in many independent copies of the human genome. As a first step toward achieving this goal, high-density oligonucleotide arrays were used in combination with somatic cell genetics to identify a large fraction of all common human chromosome 21 SNPs and to observe directly the SNP haplotype block and SNP haplotype pattern structure defined by these SNPs.

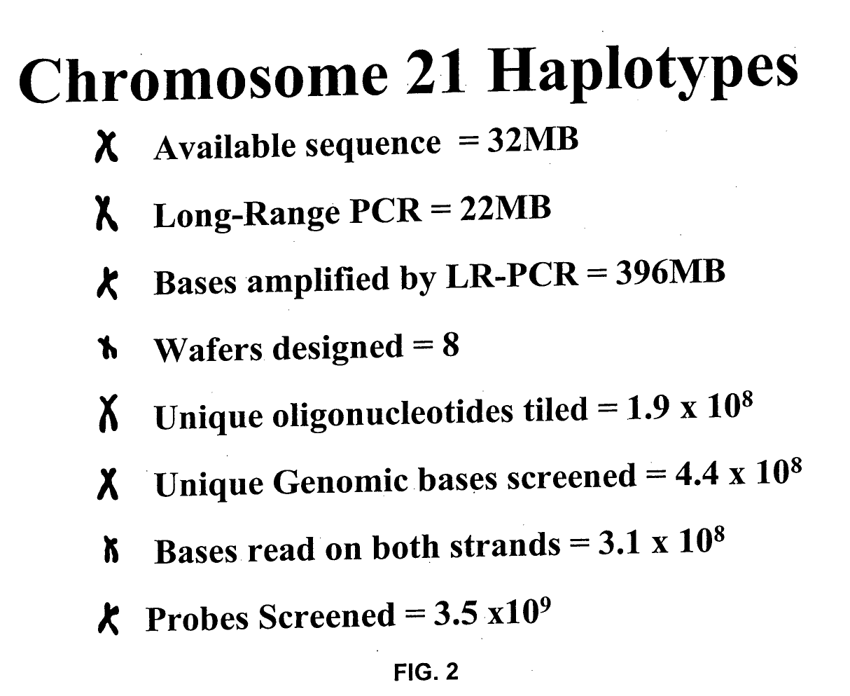

SNPs were discovered by using a publicly available panel of twenty four ethnically diverse individuals (D. E. Reich et al., Nature 411, 199 (2001)). The two copies of chromosome 21 from each individual were physically separated using a rodent-human somatic cell hybrid technique (F. S. Collins, L. D. Brooks, and A. Chakravarti, Genome Res. 8, 1229 (1998)). Twenty independent copies of chromosome 21 were analyzed for SNP discovery and haplotype structure. This sample size and composition has greater than 98% power to detect globally distributed alleles present at a frequency of 10% or greater, and 74% power to detect globally distributed alleles with a frequency of 1% or greater. Finished human chromosome 21 genomic DNA sequence consisting of 32,397,439 bases was masked for repetitive sequences, and the resulting 21,676,868 bases (67%) of unique sequence were assayed for variation using high density oligonucleotide arrays. In total, 3.4×10⁹ oligonucleotides were synthesized on 160 wafers to scan 20 independent copies of human chromosome 21 for DNA sequence variation (see FIG. 2).

Each unique chromosome 21 was amplified from a rodent-human hybrid cell line by using long range PCR. Unique oligonucleotides were designed to generate 3253 minimally overlapping PCR products of 10 kb average length spanning 32.4 Mb of contiguous chromosome 21 DNA. PCR products corresponding to the bases present on a single wafer were pooled and hybridized to the wafer as a single reaction. SNPs were detected as altered hybridization patterns by using a heurisitc algorithm (see D. G. Wang, et. al., Science 280 1077 (1998)). In total, 35,989 SNPs were identified in the sample of twenty chromosomes. The position and sequence of these human polymorphisms have will be deposited in GenBank in addition to being listed in dbSNP.txt of the CD ROM. Dideoxy sequencing was then used to assess a random sample of 300 of these SNPs in the original DNA samples and 97% of the SNPs assayed were confirmed.

In order to achieve this low rate of 3% false positive SNPs, stringent thresholds for SNP detection on wafers were used that resulted in a high false negative rate. Approximately 65% of all bases present on the wafers yielded data of high enough quality for use in SNP detection. This is very similar to the fraction of bases in single dideoxy sequencing reads that have quality scores high enough for reliable SNP detection.

Overall, 47% of the 53,000 common SNPs with an allele frequency of 10% or greater estimated to be present in 32.4 Mb of the human genome were identified. This compares with an estimate of 18-20% of all such common SNPs present in the collection generated by the International SNP Mapping Working Group and the SNP Consortium (G. Marth et al., Nature Genet. 27, 371 (2001)). The difference in coverage is explained by the fact that a larger number of chromosomes for SNP discovery were used for the present invention. In order to assess the replicability of the findings, SNP discovery was performed for one wafer design with nineteen additional copies of chromosome 21 derived from the same diversity panel as the original sample. A total of 7188 SNPs were identified using the two samples. On average, 66% of all SNPs found in one sample were discovered in a second sample of globally diverse chromosomes. As expected, failure of a SNP to replicate in a second sample is strongly dependent on allele frequency. It was found that 80% of SNPs with a minor allele present two or more times in a sample were also found in a second sample, while only 32% of SNPs with a minor allele present a single time were found in a second sample. These findings suggest that the 24,386 SNPs with a minor allele represented more than once are highly replicable in different global samples and that this set of SNPs is useful for defining common SNP haplotype patterns.

In addition to the replicability of SNPs in different samples, the distance between adjacent SNPs in a collection of SNPs is critical for defining meaningful haplotype structure. SNP haplotype blocks, which can be as short as several kb, may contain only one SNP and would go unrecognized if the distance between adjacent SNPs is large relative to the size of the actual SNP haplotype blocks. The observed SNPs on chromosome 21 were very evenly distributed across the chromosome, even though repeat sequences were not included in the SNP discovery process. The average distance between adjacent SNPs is 900 bases when all SNPs are considered, and 1300 bases if considering only the 24,386 common SNPs on chromosome 21. For this set of common SNPs, 93% of intervals between adjacent SNPs in genomic DNA, including repeated DNA, are 4000 bases or less.

The construction of haplotypes from diploid data is complicated by the fact that the relationship between alleles at any two heterozygous SNPs is not directly observable. Current methods used to circumvent this problem include statistical estimation of haplotype frequencies, direct inference from family data, and allele-specific PCR amplification over short segments. In an effort to avoid the uncertainty and missing information inherent in all of these methods, SNPs on haploid copies of chromosome 21 isolated in rodent-human somatic cell hybrids were characterized, which allows for direct determination of the full haplotypes of these chromosomes.

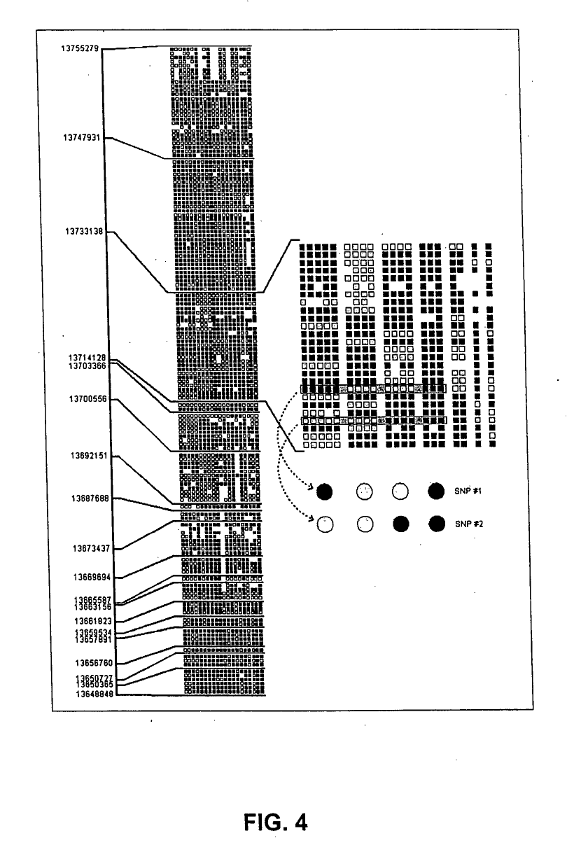

A set of 24,386 SNPs with a minor allele represented more than once in the data set was used to define the SNP haplotype block and SNP haplotype pattern structure. FIG. 4 shows the SNP haplotype patterns on twenty different chromosomes for 147 SNPs spanning a 106 kb region of human chromosome 21. Each row of colored boxes represents a single SNP. The blue boxes in each row represent the major allele for that SNP, and the yellow boxes represent the minor allele. Absence of a box at any position in a row indicates missing data. Each column of colored boxes represents a single chromosome, with the SNPs arranged in their physical order on the chromosome. Non-variant bases between adjacent SNPs are not represented in the figure. The 147 SNPs are divided into eighteen blocks, defined by black horizontal lines. The position of the base in chromosome 21 genomic DNA sequence defining the beginning of one block and the end of the adjacent block is indicated by the numbers to the left of the vertical black line. The expanded boxes on the right of the figure represent a SNP block defined by 26 common SNPs spanning 19 kb of genomic DNA. Of the seven different haplotype patterns represented in the sample, the four most common patterns include sixteen of the twenty chromosomes sampled (ie. 80% of the sample).

The allele patterns of two SNPs which distinguish unambiguously between the four common haplotypes in this block are indicated by the blue and yellow circles. Visual inspection of these data shows that although no two chromosomes share an identical SNP haplotype pattern for all 147 SNPs, there are numerous regions in which multiple chromosomes share a common SNP haplotype pattern. One such region, defined by 26 SNPs spanning 19 kb, is expanded for more detailed analysis. This block of 26 SNPs defines seven unique SNP haplotype patterns in twenty chromosomes. Despite the fact that some data is missing due to failure to pass the threshold for data quality, in all cases a given chromosome can be assigned unambiguously to one of the seven SNP haplotype patterns.

The four most frequent SNP haplotype patterns, each of which is represented by three or more chromosomes, account for 80% of all chromosomes in the sample. Furthermore, only two SNPs out of the total of twenty-six are required to completely distinguish the four most frequent SNP haplotype patterns from one another. It is remarkable that 80% of the haplotype structure of the entire global sample can be unambiguously defined by less than 10% of the total SNPs in the block. Several different possibilities exist in which three SNPs can be chosen so that each of the four common SNP haplotype patterns is defined uniquely by a single SNP. One of these “three SNP” choices would be preferred over the two SNP combination in an experiment involving pooled samples, since the two SNP combination would not unambiguously distinguish the four common SNP haplotype patterns in such a situation. In summary, while the particular situation may dictate the specific SNPs selected to capture SNP haplotype pattern information, it is clear that the majority of the SNP haplotype pattern information in the sample is contained in a very small subset of all the SNPs. It is also clear that random selection of two or three SNPs from this block of SNPs will rarely provide enough information to uniquely assign a chromosome to one of the four common haplotypes.

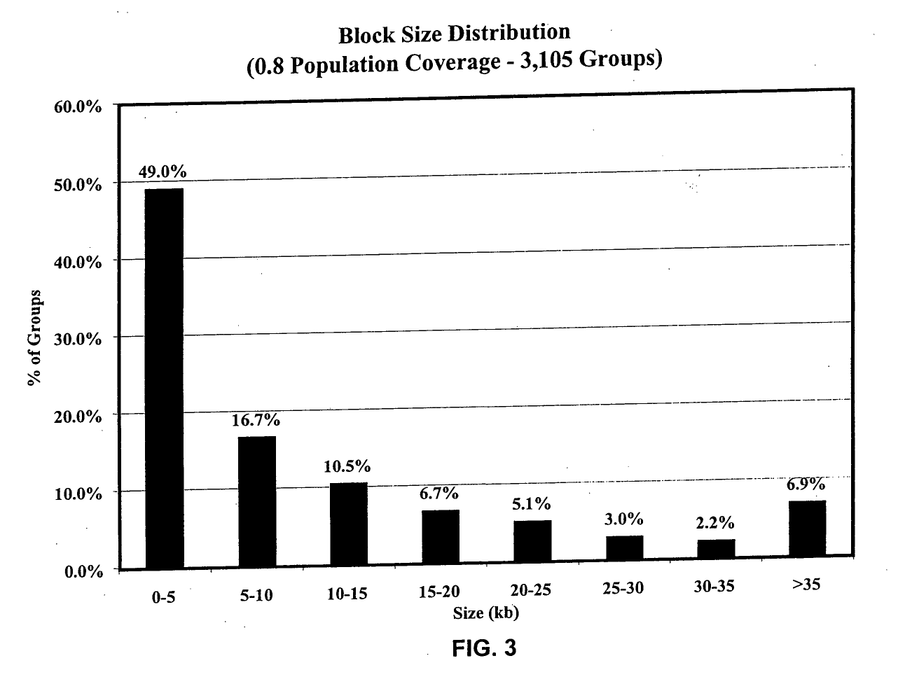

The present invention defines a set of contiguous SNP haplotype blocks spanning the entire 32.4 Mb of chromosome 21 while minimizing the total number of SNPs required to define the haplotype structure (see group.txt and pattern.txt). A heuristic approach employing a greedy algorithm was used to address this problem. All possible SNP haplotype blocks of physically contiguous SNPs of size one SNP or larger were considered. SNP haplotype blocks in which less than 80% of the chromosomes in the sample were defined by haplotypes represented more than once in the block were excluded from the analysis. Of the remaining SNP haplotype blocks, the SNP haplotype blocks with the maximum ratio of total SNPs in the SNP haplotype blocks to the minimal number of SNPs required to uniquely discriminate SNP haplotype patterns represented more than once in the SNP haplotype blocks and accounting for at least 80% of the chromosomes were selected. Any of the remaining SNP haplotype blocks that physically overlap with the selected SNP haplotype block were discarded, and the process is repeated until a set of contiguous, non-overlapping blocks that cover the 32.4 Mb of chromosome 21 with no gaps was selected. Applying this algorithm to the data set of 24,386 common SNPs, 4135 SNP haplotype blocks spanning chromosome 21 were selected. A total of 606 SNP haplotype blocks, comprising 15% of all SNP haplotype blocks on chromosome 21, contain greater than ten SNPs per block and include 44% of the total 32.4 Mb. In contrast, 2119 SNP haplotype blocks, comprising 51% of all blocks, contain less than three SNPs per block and make up only 19% of the physical length of the chromosome. Overall, the average physical size of a SNP haplotype block is 7.8 kb (see FIG. 3.) FIG. 3 shows the distribution of lengths in kilobases for the SNP haplotype blocks. Note that 49% of groups are in the 0-5 kb range. The size of a SNP haplotype block is not correlated with its order on the chromosome, and large SNP haplotype blocks are interspersed with small SNP haplotype blocks along the length of the chromosome. For those blocks containing three or more SNPs, there are an average of 2.7 common SNP haplotype patterns per SNP haplotype block, defined as SNP haplotype patterns that are observed on multiple chromosomes and together include more than 80% of the sample.

On average, the most common SNP haplotype pattern in a SNP haplotype block is represented by nine chromosomes out of the twenty chromosomes in the sample, the second most common SNP haplotype pattern is represented by four chromosomes, and the third most common SNP haplotype pattern, if present, is represented by two chromosomes. One chromosome per SNP haplotype block, on average, cannot be assigned to a unique SNP haplotype pattern due to missing data. The fact that such a large fraction of globally diverse chromosomes are represented by such limited SNP haplotype pattern diversity is remarkable. These findings are consistent with the observation that when haplotype frequency is considered, 82% of the haplotypes observed in a collection of 313 human genes are observed in all ethnic groups, while only 8% of haplotypes are population specific (L. Excoffier and M. Slatkin, Mol. Biol. Evol. 12, 921 (1995)).

In an effort to determine the functional significance, if any, of the SNP haplotype blocks defined by the methods of the present invention, the characteristics of SNP haplotype blocks containing only genic DNA was compared to SNP haplotype blocks containing only nongenic DNA. Genic DNA was defined to include all genomic DNA beginning 10 kb 5′ of the first exon of each known chromosome 21 gene and extending 10 kb 3′ of the last exon of that gene. By this definition, 39% of the 32.4 Mb of chromosome 21 analyzed was genic. It should be noted that by using this definition, many genes occur in clusters, not separated by any intervening nongenic DNA. The average SNP haplotype block size for the 1413 SNP haplotype blocks containing only genic DNA and the 2510 SNP haplotype blocks containing only nongenic DNA is not significantly different from the overall average SNP haplotype block size of 7.8 kb. In contrast, the average size of 212 SNP haplotype blocks that each contain both genic and nongenic DNA is 20.1 kb, more than two times greater than the overall average SNP haplotype block size (p<0.00001 by single factor ANOVA). For this subset of 212 “mixed” SNP haplotype blocks, increased size was observed for SNP haplotype blocks containing small numbers of SNPs as well as SNP haplotype blocks containing large numbers of SNPs. In general, a mixed SNP haplotype block contains a single boundary between genic and nongenic DNA, and this boundary is as likely to occur at the 3′ end as at the 5′ end of a genic region.

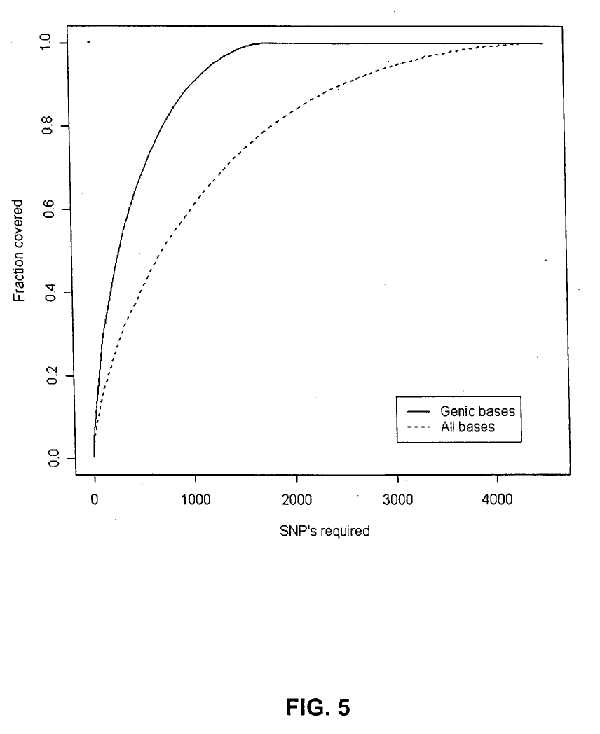

Based on knowledge of the SNP haplotype pattern structure within SNP haplotype blocks, select subsets of the 24,386 common SNPs may be selected to capture any desired fraction of the common SNP haplotype pattern information, defined as complete information for SNP haplotype patterns present more than once and including greater than 80% of the sample across the entire 32.4 Mb (FIG. 5). FIG. 5 depicts the number of SNPs required to capture the common haplotype information for chromosome 21. For each SNP block, the minimum number of SNPs required to distinguish unambiguously haplotypes in that block which are present more than once and which describe more than 80% of the chromosomes sampled was determined (ie. common haplotype information). These SNPs provide common haplotype information for the fraction of the total physical distance defined by that block. Beginning with the SNPs that provide common haplotype information for the greatest physical distance, the cumulative increase in physical coverage (i.e., fraction covered) is plotted relative to the number of SNPs added (i.e., SNPs required). For example, while a minimum of 4563 SNPs are required to capture all the common SNP haplotype pattern information, only 2793 SNPs are required to capture the common SNP haplotype pattern information in blocks containing three or more SNPs and which cover 81% of the 32.4 Mb. A total of 1794 SNPs are required to capture all the common SNP haplotype pattern information in genic DNA.

The present invention has particular relevance for whole-genome association studies mapping common disease genes, the approach relying on the hypothesis that common genetic variants are responsible for susceptibility to common diseases. By comparing the frequency of genetic variants in unrelated cases and controls, genetic association studies identify specific SNP haplotype patterns in the human genome that play important roles in disease. The recent availability of the human DNA sequence offers the possibility of surveying the entire genome, dramatically increasing the power of genetic association analysis; however, a major limitation to the implementation of this method has been lack of knowledge of the haplotype structure of the human genome, which is required in order to select the appropriate genetic variants for analysis. The unpredictable nature of haplotype structure in any particular genomic region demands a comprehensive, empiric approach to obtain the required information.

High-density oligonucleotide arrays in combination with somatic cell genetic sample preparation provide a rapid, high-resolution approach to defining empirically the common SNP haplotype pattern structure of the human genome. Although the length of genomic regions with a SNP haplotype block is extremely variable, a dense set of common SNPs enables systematic approach to define SNP haplotype blocks of the human genome in which 80% of the global human population is described by only three common SNP haplotype patterns. In general, when applying the greedy algorithm, the most common SNP haplotype pattern in any SNP haplotype block is found in 50% of individuals, the second most common in 25% of individuals, and the third most common in 12.5% of individuals.

It is important to note that SNP haplotype blocks are defined based on their genetic information content and not on knowledge of how this information originated or why it exists. As such, SNP haplotype blocks do not have absolute boundaries, and may be defined in different ways, depending on the specific application. The heuristic algorithm provides only one of many possible approaches. The fact that the physical density of the common SNPs used to define the SNP haplotype pattern structure is six times denser than the physical size of the average SNP haplotype block of limited SNP haplotype pattern diversity indicates that a useful global haplotype map of the human genome is an achievable goal. Nevertheless, the results indicate that a very dense set of SNPs is required to capture all the common SNP haplotype pattern information. Once in hand, however, this information can be used to identify much smaller subsets of SNPs useful for comprehensive whole-genome association studies. Based on the results with chromosome 21, it is estimated that 300,000 SNPs selected with prior knowledge of common haplotype structure across the genome provides a powerful general reagent for whole-genome association analysis.

The present invention provides SNP sequences, SNP haplotype blocks and SNP haplotype patterns for human chromosome 21. In addition, the SNP haplotype patterns were analyzed to identify informative SNPs. The informative SNPs can be used to dissect the genetic bases of disease and drug response in a practical and cost effective manner unknown previously. It is to be understood that the above description is intended to be illustrative and not restrictive. Many embodiments will be apparent to those skilled in the art upon reviewing the above description. The scope of the invention should, therefore, be determined not with reference to the above description, but should instead be determined with reference to the appended claims, along with the full scope of equivalents to which such claims are entitled.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A nucleic acid segment of between 10 and 201 contiguous bases from dbSNP.txt, wherein said nucleic acid segment includes either base at a SNP locus indicated in dbSNP.txt.

2. A complement of said nucleic acid segment of claim 1.

3. A SNP locus-specific oligonucleotide that hybridizes to said nucleic acid segment of claim 1.

4. A haplotype block from human chromosome 21 as described in group.txt.

5. A haplotype pattern from human chromosome 21 as described in pattern.txt.

6. A method of analyzing a nucleic acid from an individual comprising: obtaining said nucleic acid and identifying a base occupying any one of said SNP loci indicated in dbSNP.txt.

7. The method of claim 6, wherein said nucleic acid is obtained from a plurality of individuals, and said base occupying any one of said SNP loci is determined in each of said plurality of individuals.

8. The method of claim 7, further comprising testing each of said plurality of individuals for a presence of a disease phenotype and correlating said presence of said disease phenotype with said base.

9. A computer-readable storage medium for storing data for access by an application program being executed on a data processing system comprising: a data structure stored in said computer-readable storage medium, said data structure including information resident in a database used by said application program and including a plurality of records, each record of said plurality comprising information identifying a SNP locus indicated in dbSNP.txt.

10. The computer-readable storage medium of claim 9, wherein each record has a field identifying a base occupying said SNP locus and a location of said SNP locus.

11. The computer-readable storage medium of claim 10, wherein each record identifies a nucleic acid segment of between 10 and 100 bases from a sequence from dbSNP.txt including a SNP locus indicated therein, or the complement of said nucleic acid segment.

12. A signal carrying data for access by an application program being executed on a data processing system comprising: a data structure encoded in said signal, said data structure including information resident in a database used by said application program and including a plurality of records, each record of the plurality comprising information identifying a SNP locus from dbSNP.txt.
